(19) United States
(12) Patent Application Publication
(10) Pub. No.: US 2005/0187417 A1
(43) Pub. Date: Aug. 25, 2005

(54) Process for the production of phenylalkanes using a hydrocarbon fraction that is obtained from the Fischer-Tropsch process

(76) Inventors: Patrick Briot, Pommier de Beaurepaire (FR); Emmanuelle Guillon, Saint Genis Laval (FR); Francois Hugues, Vernaizon (FR); Marie-Claire Marion, Vernaison (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/061,573

(22) Filed: Feb. 22, 2005

(30) Foreign Application Priority Data
Feb. 19, 2004 (FR) 04/01.746

(51) Int. Cl. C07C 1/20; C07C 2/68

(52) U.S. Cl. 585/469; 585/467

(57) ABSTRACT

A process for the production of phenylalkanes comprising a reaction for alkylation of at least one aromatic compound by at least one hydrocarbon fraction that is directly obtained from the Fischer-Tropsch process comprising linear olefins that have 9 to 16 carbon atoms per molecule and oxygenated compounds is described. Said alkylation reaction is carried out in a catalytic reactor that contains at least one reaction zone that comprises at least one acidic solid catalyst, and said hydrocarbon fraction does not undergo any purification treatment prior to its introduction into said reaction zone.

The first process, described in, for example, the Ullman's Encyclopedia, 5^{th} Volume A 25, page 766, uses, during the benzene alkylation stage, hydrofluoric acid as an acid catalyst.

The second process, described in, for example, Ullmann's Encyclopedia, 5^{th} Volume A 25, page 766, uses a Friedel and Craft-type catalyst, generally based on AlCl₃.

These two catalyst types were used in solution state in the reaction medium.

These two processes lead to the formation of phenylalkane isomers, for example 2-, 3-, 4-, 5-, and 6-phenylalkane isomers.

The primary drawback of these processes is linked to environmental constraints.

The first process, based on the use of hydrofluoric acid, poses safety problems, on the one hand, and waste treatment problems, on the other hand.

The second process poses the problem of wastes that are obtained from the use of said Friedel and Craft-type catalysts. Actually, it is necessary in this case to neutralize the effluents by a basic solution at the reactor outlet. In addition, the separation of the catalyst from the products of the reaction is necessary and difficult to use for the two processes.

### PRIOR ART

To solve these drawbacks, an alkylation of benzene by linear olefins in the presence of a solid catalyst was proposed.

The prior art notes, for example, the use of catalysts that have geometric selectivity properties and that lead to an improved selectivity of 2- and 3-phenylalkanes. The catalysts that exhibit geometric selectivity properties generally consist of zeolitic compounds as defined in the classification “Atlas of Zeolite Structure Types,” W. M. Meier, D. H. Olson and Ch. Baerlocher, 5^{th} Revised Edition, 2001, Elsevier to which this application also refers. U.S. Pat. No. 4,301,317 thus proposes a series of zeolites, including cancrinite, gmelinite, mordenite, offretite and ZSM-12.

Patent Application FR-A-2 697 246 proposes using catalysts based on dealuminified Y zeolite.

In contrast, Patent Application EP-A-160 144 discloses the use of Y zeolites whose crystallinity varies from 30 to 80%, while U.S. Pat. No. 5,036,033 teaches the use of Y zeolites that are rich in ammonium cations.

The production of phenylalkanes is done by alkylation of benzene with linear olefins that are obtained mainly for the dehydrogenation of linear paraffins that are obtained from a kerosene fraction such as the one that is described in U.S. Pat. No. 6,417,420 and U.S. Pat. No. 6,479,720. These fractions contain in general between 8 and 12% by weight of dilute olefins in paraffins (between 88 and 92% by weight). This content is limited by the thermodynamic equilibrium of the reaction for dehydrogenation of the paraffins concerned.

The arrangement relying on linear paraffins that are obtained from a kerosene fraction is complex and costly since it also comprises the dehydrogenation of paraffins that make it possible to obtain olefins, whereby a selective hydrogenation makes it possible to reduce into mono-olefins the diolefins that are formed during the dehydrogenation stage of the paraffins.

A fraction that is obtained from the atmospheric distillation of the crude and that distills in the range of 150° C. to 240° C. is called a kerosene fraction.

The purpose of this invention is to propose a process for the production of phenylalkanes whose use is simplified.

### BRIEF PRESENTATION OF THE INVENTION

It was found that the use of a C9-C16 hydrocarbon fraction, preferably a C10-C14 hydrocarbon fraction, directly obtained from the Fischer-Tropsch process, as an olefinic feedstock for the alkylation of at least one aromatic compound, preferably benzene, led, surprisingly enough, to a higher production of linear alkylbenzenes than the one obtained by using an olefinic feedstock obtained from the dehydrogenation of linear paraffins and that the use of such a feedstock also made possible a simplified implementation of the process for the production of linear alkylbenzenes.

This invention also has as its object a process for the production of phenylalkanes comprising a reaction for alkylation of at least one aromatic compound by at least one hydrocarbon fraction that is directly obtained from the Fischer-Tropsch process and that comprises linear olefins having 9 to 16 carbon atoms per molecule and oxygenated compounds, whereby said reaction is called out in a catalytic reactor that contains at least one reaction zone that comprises at least one acidic solid catalyst, whereby said hydrocarbon fraction has not undergone any purification treatment prior to its introduction into said reaction zone. Said acidic solid catalyst preferably comprises at least one zeolite.

### DETAILED DESCRIPTION OF THE INVENTION

This invention has as its object a process for the production of phenylalkanes comprising a reaction for alkylation of at least one aromatic compound by at least one hydrocarbon fraction that is directly obtained from the Fischer-Tropsch process comprising linear olefins that have 9 to 16 carbon atoms per molecule and oxygenated compounds, whereby said reaction is carried out in a catalytic reactor that contains at least one reaction zone that comprises at least one acidic solid catalyst, whereby said hydrocarbon fraction has not undergone any purification treatment prior to its introduction into said reaction zone.

According to the process according to the invention, the aromatic compound is preferably benzene or toluene, preferably benzene. The hydrocarbon fraction that is directly obtained from the Fischer-Tropsch process comprises linear olefins that preferably have 10 to 14 carbon (C10-C14) atoms per molecule.

The process developed by the applicant proposes using a C9-C16 hydrocarbon fraction or preferably a C10-C14 hydrocarbon fraction that is directly obtained from the Fischer-Tropsch process. The Fischer-Tropsch process constitutes one of the stages that make it possible to produce synthetic fuels from a synthesis gas (GTL chain). The Fischer-Tropsch synthesis, used in the Fischer-Tropsch process, is a polymerization reaction that, according to the catalyst the operating conditions used, can lead to the formation of paraffinic- or olefinic-type hydrocarbons and also oxygenated compounds (in particular linear alcohols) in various proportions.

The products that are obtained from the Fischer-Tropsch synthesis cover a broad range in terms of carbon number n, including methane (n=1) up to products with very long carbon chains (n≧80). In particular, the Fischer-Tropsch process makes it possible to produce light C9-C16 fractions that are for the most part paraffinic but that also contain olefins, primarily alpha olefins, whose concentration is particularly well suited for the production of phenylalkanes, according to this invention.

For example, by way of illustration, it is possible to obtain via the Fischer-Tropsch process a C9-C16 fraction that contains about 10 to 25%, preferably 14 to 25%, and very preferably 17 to 23%, by weight of olefins, whose proportion of alpha linear olefins can be greater than 65%, and a C10-C14 fraction that contains about 10 to 30%, preferably 14 to 28%, and very preferably 17 to 25%, by weight of olefins, whose proportion of alpha linear olefins can be greater than 63%. Such a hydrocarbon fraction can be used directly for the implementation of the process according to the invention.

These fractions, however, also contain oxygenated compounds, in particular alcohols that are generally linear and whose concentration can be between 2 and 7% by weight.

During the alkylation reaction of at least one aromatic compound, preferably benzene, these oxygenated compounds will dehydrate and generate water that is a poison of acid catalysts during alkylation reactions. This impact of the water is well described by W. Liang et al. (Zeolites 17, pp. 297-303 (1996)).

To avoid this problem, it is necessary to eliminate the oxygenated compounds and in particular the alcohols, in particular the linear alcohols, contained in the fractions that are obtained from the Fischer-Tropsch process before using said fractions as olefinic feedstocks in the reaction for alkylation of at least one aromatic compound, preferably benzene.

The applicant proposes a direct use of the hydrocarbon fractions that are obtained from the Fischer-Tropsch process, i.e., that have not undergone any purification treatment prior to their introduction into the reaction zone, and in particular without prior separation of oxygenated compounds. The applicant noted, surprisingly enough, that the presence of these oxygenated compounds in the hydrocarbon fraction comprising linear olefins having 9 to 16 carbon atoms, preferably 10 to 14 carbon atoms, per molecule does not impair the activity of the acidic solid catalyst.

Surprisingly enough, and thanks to a particular selection of operating conditions, in particular the temperature, the alkylation process, the object of the invention, it is possible to obtain excellent yields of phenylalkanes and to maintain a level of stability of the catalyst that is higher than the one that is observed in the processes that use conventional fractions that are obtained from kerosene.

According to the process, the object of the invention, the hydrocarbon fraction or fractions that is (are) obtained from the Fischer-Tropsch process is (are) mixed with the aromatic compound or compounds upstream from the reaction zone.

The hydrocarbon fraction that is directly obtained from the Fischer-Tropsch process and that is used as an olefinic feedstock for the alkylation of the aromatic compound or compounds, preferably benzene, for the implementation of the process according to the invention, generally contains oxygenated compounds in a proportion that represents up to 35% by weight of said fraction. Said oxygenated compounds are alcohols, aldehydes, ketones, ethers, esters and/or acids. They preferably contain the same number of carbon atoms as the linear olefins that are present in said fraction that is directly obtained from the Fischer-Tropsch process, i.e., they contain 9 to 16 carbon atoms and preferably 10 to 14 carbon atoms per molecule. Said oxygenated compounds are preferably alcohols that have 9 to 16 carbon atoms per molecule and very preferably that have 10 to 14 carbon atoms per molecule. They are advantageously linear alcohols. The fraction that is directly obtained from the Fischer-Tropsch process and that is used as a feedstock for the alkylation of the aromatic compound or compounds, preferably benzene, for the implementation of the process according to the invention has a distillation interval of 150° C. to 300° C., and preferably of 170° C. to 270° C.

The reactor that is used for the implementation of the process according to the invention is a fixed-bed reactor that works with a large excess of aromatic compound, preferably benzene, so as to limit the formation of dialkylbenzenes (DAB) and also to limit the elevation of temperature at the outlet of the reactor, whereby the reaction is exothermic.

In general, the alkylation stage is followed by at least one stage for separation of reagents that are in excess, and at least one stage for separation of monoalkylated compounds that are obtained from the reaction. These separations are carried out on one or more distilling columns. For example, and according to a preferred embodiment of the process according to the invention, the product that is obtained is fractionated at the outlet of the reaction zone, in a distilling column, so as to collect separately:

a) a first fraction that contains the aromatic compound, preferably unconverted benzene and water,

b) a second fraction that contains at least the unconverted C₉-C₁₆ (preferably C₁₀-C₁₄) linear olefin, as well as the paraffins that are optionally initially present in the feedstock, as well as the oxygenated compounds that are initially present in the fraction that is directly obtained from the Fischer-Tropsch process,

c) a third fraction containing phenylalkane isomers that constitutes all of the monophenylalkanes,

d) a fourth fraction that contains at least one poly-alkylbenzene (or polyalkylbenzene fraction), whereby the latter can be at least in part recycled to the reaction zone so as to be transalkylated (according to a transalkylation reaction), and a phenylalkane isomer mixture is collected.

The second fraction that contains at least one unconverted C₉-C₁₆ (usually C₁₀-C₁₄) linear olefin preferably can be at least in part recycled to the reaction zone.

According to the invention, the acidic solid alkylation catalyst that is used in the alkylation process can be crystallized or amorphous. It can be in particular a catalyst that consists exclusively of an amorphous phase such as silica-alumina, silicate alumina that may or may not be doped by halogens. It can also be a catalyst that consists of a clay phase, bridged or modified clay. It can also be a catalyst that comprises at least one zeolite of crystalline structure, for example that has a structure as defined in the classification “Atlas of Zeolite Framework Type,” (W. M. Meier, D. H. Olson and Ch. Baerlocher, 5^{th} Revised Edition, 2001, Elsevier).

The acidic solid catalyst preferably comprises at least one zeolite that is selected from the group that consists of the zeolites of structural types FAU, MOR, MTW, OFF, MAZ, BEA and EUO.

Among the FAU-structural-type zeolites, the Y zeolite and the Y zeolite that is exchanged with rare earths (REY) are preferred. Among the MOR-structural-type zeolites, the mordenite zeolite is preferred. Among the MTW-structural-type zeolites, the ZSM-12 zeolite is preferred. Among the OFF-structural-type zeolites, the offretite zeolite is preferred. Among the MAZ-structural-type zeolites, the ZSM-4 zeolite is preferred. Among the BEA-structural-type zeolites, the beta zeolite is preferred, and among the EUO-structural-type zeolites, the EU-I zeolite is preferred.

Very preferably, the catalyst advantageously comprises at least one dealuminified Y zeolite, with an overall Si/Al atomic ratio that is more than 4, preferably between 8 and 70, and even more advantageously between 15 and 50.

The dealuminified Y zeolite is generally used mixed with a binder or a matrix that is generally selected from the group that is formed by the clays, the aluminas, silica, magnesia, zirconia, titanium oxide, boron oxide and/or any combination of at least two of these oxides such as silica-alumina or silica-magnesia. All of the known methods of agglomeration and shaping are applicable, such as, for example, extrusion, pelletizing, and drop coagulation. The catalyst that is contained in the reaction zone of the reactor generally contains 1 to 100%, preferably 20 to 98%, and very preferably 40 to 98% of said dealuninified Y zeolite, and 0 to 99%, preferably 2 to 80%, and, for example, 2 to 60% by weight of a binder or a matrix. The dealuminified Y. zeolites and their preparation are known. Reference can be made to, for example, U.S. Pat. No. 4,738,940.

The Y zeolite, which may or may not be dealuminified, used in the process according to the invention, is preferably at least partially in acid form (HY zeolite) and is characterized by different specifications:

an overall Si/Al atomic ratio that is more than 4, preferably between 8 and 70, and even more preferably between 15 and 50,

a sodium content of less than 0.25% by weight,

a crystalline parameter of the elementary mesh of less than 24.55.10⁻¹⁰ m and preferably between 24.20.10⁻¹⁰ m and 24.39.10⁻¹⁰ m,

a specific surface area that is determined by the B.E.T. method of more than about 300 m²/g and preferably more than about 450 m²/g,

a water vapor adsorption capacity at 25° C. for a partial pressure of 3.46 mbar (millibar), more than about 0.5% and preferably more than about 3%.

The dealuminified Y zeolites are synthesized, for example, generally from any NaY zeolite, by a suitable combination of two basic treatments: (a) a hydrothermic treatment that associates temperature, whereby the temperature is preferably between 450 and 900° C. and very preferably between 550 and 800° C., and water vapor partial pressure (40 to 100% of water vapor), and (b) an acid treatment by, preferably, a strong and concentrated mineral acid (0.01 to 10 N). Stage (a) is only optional, however. In general, the NaY zeolite, starting from which the Y zeolite that is used in the catalyst that is present in the reaction zone is prepared, has an overall Si/Al atomic ratio of between about 1.8 and 3.5; it would be advisable first to lower its content by weight of sodium to less than 3% and preferably less than 2.5%. hie reduction of the sodium content can be carried out by ion exchanges of the NaY zeolite in ammonium salt solutions (nitrate, sulfate, oxalate, etc.) with an ammonium concentration of between 0.01 and 10 N, at a temperature of between 10 and 180° C. (exchange optionally under autogenous pressure), for a duration that is more than about 10 minutes. The NaY zeolite also has in general a specific surface area of between about 750 and 950 m²/g.

Another preferred method of the invention consists in using a zeolite mixture as an acidic solid catalyst in the reaction zone of the process according to the invention. It can be, for example, a zeolite mixture that consists of at least one Y zeolite as described above, and at least one MOR-structural-type zeolite, in particular a mordenite-type zeolite. The preparation of MOR-structural-type zeolites is known from the prior art (U.S. Pat. No. 4,503,023). Concerning the preparation of a catalyst that comprises a mixture of zeolites, the mixture of said zeolites, which are found in the powder state, is carried out by any powder-mixing techniques that are known to one skilled in the art and followed by shaping. When the zeolite powder mixing is completed, the mixture is shaped by any technique that is known to one skilled in the art. In particular, it can be mixed with a matrix, which is generally amorphous; for example with a wet alumina gel powder. The shaping can also be carried out with matrices other than alumina, such as, for example, magnesia, amorphous silica-aluminas, natural clays (kaolin, bentonite, sepiolite, attapulgite), silica, titanium oxide, boron oxide, zirconia, aluminum phosphates, titanium phosphates, zirconium phosphates, carbon and mixtures thereof. Mixtures of alumina and silica, and mixtures of alumina and silica-alumina can also advantageously be used. It is preferred to use matrices that contain alumilna in all of its forms that are known to one skilled in the art, and even more preferably gamma-alumina. The mixture is then shaped. Several techniques can be used for this purpose and in particular the extrusion through a dye, pelletizing and tabletting. The zeolite mixture can also consist of a mixture of zeolites that were already shaped as described above.

The catalyst that is used for the implementation of the process according to the invention is shaped in the form of grains of various shapes and sizes. It is used in general in the form of cylindrical extrudates or multilobar extrudates, such as bilobar, trilobar, or multilobar extrudates of straight or twisted shape, but can optionally be manufactured and used in the form of crushed powder, tablets, rings, balls or disks.

After the shaping stage, the product that is obtained is subjected to a drying stage at a temperature of between 100 and 300° C., preferably between 120 and 200° C., then to a calcination stage at a temperature of between 350 and 650° C., preferably between 450 and 600° C.

The operating conditions that are applied in the reaction zone are selected, of course, by one skilled in the art based on the nature of the catalyst. Usually, for the reactions for alkylation of benzene, the temperature is between 80° C. and 180° C. This was extensively described in the literature (Applied Catalysis A: General 184, pp. 231-238 (1999); Applied Catalysis A: General 238, pp. 99-107 (2003)). Taking into account the specificity of the olefinic feedstock that is used for the alkylation, the alkylation reaction is carried out at a temperature of more than 180° C., and preferably more than 200° C. The total pressure is between 1 and 10 MPa, preferably between 2 and 6 MPa, with a flow rate of liquid hydrocarbons (volumetric flow rate) of about 0.5 to 50 volumes per volume of catalyst and per hour, and a benzene/olefin molar ratio of between 1 and 40.

The invention will be better understood from reading the following examples.

### EXAMPLE 1

### Alkylation of Benzene by a Fraction that is Obtained from the Dehydrogenation of a Kerosene (Under Standard Conditions: Example for Comparison)

A reactor that contains 50 cm³ of catalyst that consists of 40% dealuminified Y zeolite, with an Si/Al ratio that is close to 45 in extrudate form, is used.

The operating conditions for the alkylation of benzene and a petroleum fraction obtained from the dehydrogenation of a kerosene (fraction that is obtained from the atmospheric distillation of the crude and that has a distillation range of between 150° C. and 240° C.) are standard operating conditions that are known to one skilled in the art:

temperature: 135° C.

pressure: 4 MPa

VVH=1 h⁻¹

benzene/olefin molar ratio: 10/1

A feedstock that contains 32% by weight of benzene and 68% by weight of kerosene fraction that is obtained from the dehydrogenation is prepared. The composition of this fraction is presented in Table 1 below:

TABLE 1
Composition of the Fraction that is Obtained from the
Dehydrogenation of Kerosene.

| | Olefins | Paraffins |
|---|---|---|
| Carbon Atom | (% by Weight) | (% by Weight) |
| C10 | 1.67 | 15.02 |
| C11 | 3.29 | 29.59 |
| C12 | 2.81 | 25.31 |
| C13 | 2.23 | 20.08 |

At the outlet of the reaction zone, the products are collected, and the composition of the effluents presented in Table 2 below is obtained.

TABLE 2
Composition By Weight of the Effluents of the Reaction.

| Compounds | Content By Weight (%) |
|---|---|
| Benzene | 28.6 |
| Paraffins | 61.2 |
| C10 LAB | 1.8 |
| C11 LAB | 3.4 |
| C12 LAB | 2.8 |
| C13 LAB | 2.2 |
| LAB Total | 10.2 |

A C10 LAB is a linear alkylbenzene whose alkyl chain comprises 10 carbon atoms. It is a mixture of 2-, 3-, 4-, 5-phenylalkanes whose alkyl chain comprises 10 carbon atoms. A C11 LAB is a linear alkylbenzene whose alkyl chain comprises 11 carbon atoms. It is a mixture of 2-, 3-, 4-, 5-, 6-phenylalkanes whose alkyl chain comprises 11 carbon atoms. A C12 LAB is a linear alkylbenzene whose alkyl chain comprises 12 carbon atoms. It is a mixture of 2-, 3-, 4-, 5-, 6-phenylalkanes whose alkyl chain comprises 12 carbon atoms. A C13 LAB is a linear alkylbenzene whose alkyl chain comprises 13 carbon atoms. It is a mixture of 2-, 3-, 4-, 5-, 6-, 7-phenylalkanes whose alkyl chain comprises 13 carbon atoms.

### EXAMPLE 2

### Alkylation of Benzene by, a Fischer-Tropsch Fraction According to the Invention

A reactor containing 50 cm³ of catalyst that consists of 40% dealuminified Y zeolite, with an Si/Al ratio that is close to 45 in extrudate form, is used.

The fraction that is used in this example for the alkylation of benzene was selected so as to have the same number of carbon atoms as the fraction that is obtained from the dehydrogenation of the kerosene of Example 1, i.e., a C10-C13 fraction that is obtained from the Fischer-Tropsch process. The composition of this fraction is presented in Table 3.

TABLE 3
Composition By Weight of the Fischer-Tropsch Fraction

| Carbon Atom | Paraffins | Olefins | Alcohols |
|---|---|---|---|
| Number | (% by Weight) | (% by Weight) | (% by Weight) |
| C10 | 17.8 | 7.1 | 1.4 |
| C11 | 18.6 | 5.9 | 1.4 |
| C12 | 18.7 | 4.8 | 1.4 |
| C13 | 18.2 | 3.7 | 1 |
| Total | 73.3 | 21.5 | 5.2 |

The fraction that is obtained from the Fischer-Tropsch process has an olefin content of more than that of the fraction that is obtained from the dehydrogenation of a kerosene fraction.

In the same way as above, the feedstock is mixed with benzene such that the benzene/olefin molar ratio at the inlet of the reaction zone is close to 10 mol/mol.

This feedstock is injected into the reaction zone under the following operating conditions:

temperature: 230° C.

pressure: 4 MPa

VVH=1 h⁻¹

benzene/olefin molar ratio: 10/1

At the outlet of the reaction zone, the products are collected, and the composition of the effluents that is presented in Table 4 is obtained.

TABLE 4
Composition By Weight of the Products of the Reaction.

| Compounds | Content By Weight (%) |
|---|---|
| Benzene | 45.5 |
| Paraffins | 35.3 |
| Water | 0.3 |
| C10 LAB | 6.3 |
| C11 LAB | 5.2 |
| C12 LAB | 4.2 |
| C13 LAB | 3.2 |
| LAB Total | 18.9 |

A C10 LAB is a linear alkylbenzene whose alkyl chain comprises 10 carbon atoms. It is a mixture of 2-, 3-, 4-, 5-phenylalkanes whose alkyl chain comprises 10 carbon atoms. A C I1 LAB is a linear alkylbenzene whose alkyl chain comprises 11 carbon atoms. It is a mixture of 2-, 3-, 4-, 5-, 6-phenylalkanes whose alkyl chain comprises 11 carbon atoms. A C12 LAB is a linear alkylbenzene whose alkyl chain comprises 12 carbon atoms. It is a mixture of 2-, 3-, 4-, 5-, 6-phenylalkanes whose alkyl chain comprises 12 carbon atoms. A C13 LAB is a linear alkylbenzene whose alkyl chain comprises 13 carbon atoms. It is a mixture of 2-, 3-, 4-, 5-, 6-, 7-phenylalkanes whose alkyl chain comprises 13 carbon atoms.

Surprisingly enough, no reduction in the activity of the catalyst was noted, and in addition, the production of phenylalkanes is considerably greater than that obtained when the olefinic feedstock that is used for the alkylation is obtained from the dehydrogenation of a kerosene fraction.

1. Process for the production of phenylalkanes comprising a reaction for alkylation of at least one aromatic compound by at least one hydrocarbon fraction that is directly obtained from the Fischer-Tropsch process comprising linear olefins that have 9 to 16 carbon atoms per molecule and oxygenated compounds, whereby said reaction is carried out in a catalytic reactor that contains at least one reaction zone that comprises at least one acidic solid catalyst, whereby said hydrocarbon fraction has not undergone any purification treatment prior to its introduction into said reaction zone.

2. Process according to claim 1, in which said oxygenated compounds are present in a proportion that represents up to 35% by weight of said hydrocarbon fraction that is directly obtained from the Fischer-Tropsch process.

3. Process according to claim 1, in which said oxygenated compounds are alcohols, aldehydes, ketones, ethers, esters and/or acids.

4. Process according to claim 1, in which said oxygenated compounds are alcohols that have 9 to 16 carbon atoms per molecule.

5. Process according to claim 1, in which the aromatic compound is benzene.

6. Process according to claim 1, in which the linear olefins are olefins that have 10 to 14 carbon atoms per molecule.

7. Process according to claim 1, in which the alkylation reaction is carried out at a temperature of more than 180° C.

8. Process according to claim 7, in which the alkylation reaction is carried out at a temperature of more than 200° C.

9. Process according to claim 1, in which the acidic solid catalyst comprises at least one zeolite that is selected from the group that consists of the zeolites of the structural type FAU, MOR, MTW, OFF, MAZ, BEA or EUO.

10. Process according to claim 1, in which the acidic solid catalyst comprises at least one dealuminified Y zeolite with an overall Si/Al atomic ration of more than 4.
